(19) United States
(12) Patent Application Publication
(10) Pub. No.: US 2022/0031594 A1
(43) Pub. Date: Feb. 3, 2022

(54) COMPOSITION OF ACTIVE INGREDIENTS, FOR CARE AND SURFACE MODIFICATION OF HUMAN HAIR

(71) Applicant: Henkel AG & Co. KGaA, Duesseldorf (DE)

(72) Inventors: RENE KROHN, Norderstedt (DE); ERIK SCHULZE ZUR WIESCHE, Bielefeld (DE)

(73) Assignee: Henkel AG & Co. KGaA, Duesseldorf (DE)

(21) Appl. No.: 17/290,210

(22) PCT Filed: Oct. 31, 2019
(86) PCT No.: PCT/EP2019/079771

§ 371 Date: Apr. 29, 2021

(30) Foreign Application Priority Data
Oct. 31, 2018 (DE) 10 2018 127 254.5

(51) Int. Cl. A61K 8/58; A61K 8/41; A61Q 5/00

(52) U.S. Cl. CPC A61K 8/585; A61Q 5/00; A61K 8/41

(57) ABSTRACT

The present disclosure relates to an active ingredient composition for the care and surface modification of human hair. In particular, the present disclosure relates to a cosmetic composition for treating a keratinous material comprising a) at least one organic silicon compound and b) a cationic surfactant, the cosmetic composition being particularly suitable for caring for damaged hair.

### CROSS-REFERENCE TO RELATED APPLICATION

This application is a U.S. National-Stage entry under 35 U.S.C. § 371 based on International Application No. PCT/EP2019/079771, filed Oct. 31, 2019, which was published under PCT Article 21(2) and which claims priority to German Application No. 10 2018 127 254.5, filed Oct. 31, 2018, which are all hereby incorporated in their entirety by reference.

### TECHNICAL FIELD

The present disclosure relates to cosmetic compositions for treating a keratinous material, the composition comprising an organic silicon compound and a cationic surfactant, and the use of the cosmetic composition.

### BACKGROUND

The external exposure of hair to chemicals from a variety of different sources poses challenges for the development of cosmetic care products. Air and water impurities have a detrimental effect on skin and hair. Major air pollutants include polycyclic aromatic hydrocarbons, volatile organic compounds, nitrogen oxides (NOx), particulate matter, and cigarette smoke. The effect of various air pollutants can be enhanced in the presence of other air pollutants and when exposed to UV radiation.

It is known that the toxicity of gaseous pollutants in the air, such as sulfur dioxide, ozone, and nitrogen oxides, is related to their initiator activity for free radicals, which cause damage to living organisms. Free radicals are metabolic products that also occur naturally in the body. In large quantities, free radicals can promote irritation and inflammation and accelerate the process of aging. In this case, the term “oxidative damage” is used. Free radicals can also cause hair damage, which is visible, for example, as a reduction in shine as well as grip and/or fading of hair color.

Particulate matter is a complex mixture containing metals, minerals, organic toxins, and/or biological materials. They can also promote the formation of free radicals.

Furthermore, often changing consumer demands for a certain hair texture are associated with recurring chemical exposure of the hair. For example, hair coloring stresses the hair, due to which a special, intensive care may be necessary.

In the prior art, organosilicon compounds from the group of silanes comprising at least one hydroxy group and/or hydrolyzable group are described. Due to the presence of the hydroxy groups and/or hydrolyzable groups, the silanes are reactive substances that hydrolyze or oligomerize or polymerize in the presence of water. The oligomerization or polymerization of the silanes initiated by the presence of the water, when applied to a keratinous material, ultimately leads to the formation of a film that can exert a protective effect.

There is a need for a product that can reduce or prevent the harmful effects of air and water contaminants on a keratinous material, especially hair, or the stressful effects caused by hair treatments on a keratinous material, especially hair.

### BRIEF SUMMARY

Disclosed is a cosmetic composition for treating a keratinous material comprising at least one organic silicon compound and a cationic surfactant.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The task underlying the present disclosure is to provide a product with an improved care and/or protection effect. In particular, the present disclosure was based on the task of providing a cosmetic agent which, when used, offers increased acceptance, especially in terms of softness of the hair.

This task is solved by a cosmetic agent for the treatment of a keratinous material, comprising

a) at least one organic silicon compound and

b) a cationic surfactant.

By a keratinous material is meant hair, the skin, the nails (such as fingernails and/or toenails). Wool, furs, and feathers also fall under the definition of keratinous material.

In an exemplary embodiment, a keratinous material is understood to mean human hair, human skin, and human nails, in particular fingernails and toenails. For example, keratinous material is understood to mean human hair, in particular head and/or beard hair.

As a first ingredient essential to the invention, the cosmetic composition for treating a keratinous material contains at least one organic silicon compound. Suitable organic silicon compounds are selected from silanes having one, two or three silicon atoms, wherein the organic silicon compound includes one or more hydroxyl groups and/or hydrolyzable groups per molecule.

Organic silicon compounds, alternatively called organosilicon compounds, are compounds which either have a direct silicon-carbon bond (Si—C) or in which the carbon is bonded to the silicon atom via an oxygen, nitrogen, or sulfur atom. The organic silicon compounds are compounds containing one to three silicon atoms. Organic silicon compounds in an exemplary embodiment contain one or two silicon atoms.

According to IUPAC rules, the term silane stands for a group of chemical compounds based on a silicon skeleton and hydrogen. In organic silanes, the hydrogen atoms are completely or partially replaced by organic groups such as (substituted) alkyl groups and/or alkoxy groups. In organic silanes, some of the hydrogen atoms may also be replaced by hydroxy groups.

The agent for treating a keratinous material contains at least one organic silicon compound in an exemplary embodiment selected from silanes having one, two or three silicon atoms, wherein the organic silicon compound includes one or more hydroxyl groups or hydrolyzable groups per molecule.

In a most suitable embodiment, the agent for treating a keratinous material includes at least one organic silicon compound selected from silanes having one, two or three silicon atoms, wherein the organic silicon compound further includes one or more basic groups and one or more hydroxyl groups or hydrolyzable groups per molecule.

This basic group can be, for example, an amino group, an alkylamino group or a dialkylamino group, which is in an exemplary embodiment connected to a silicon atom via a linker. The basic group is in an exemplary embodiment an amino group, a C₁-C₆ alkylamino group or a di(C₁-C₆)alkylamino group.

The hydrolyzable group(s) is (are) in an exemplary embodiment a C₁-C₆ alkoxy group, especially an ethoxy group or a methoxy group. It is suitable when the hydrolyzable group is directly bonded to the silicon atom. For example, if the hydrolyzable group is an ethoxy group, the organic silicon compound in an exemplary embodiment contains a structural unit R′R″R′″SiO—CH₂—CH₃. The residues R′, R″ and R′″ represent the three remaining free valences of the silicon atom.

Particularly good results were obtained when the agent for treating a keratinous material contains at least one organic silicon compound of formula (I) and/or (II).

The compounds of formulae (I) and (II) are organic silicon compounds selected from silanes having one, two or three silicon atoms, the organic silicon compound comprising one or more hydroxyl groups and/or hydrolysable groups per molecule.

In another very particularly suitable embodiment, the agent for treating a keratinous material includes at least one organic silicon compound of formula (I) and/or (II),

R₁R₂N-L-Si(OR₃)ₐ(R₄)_{b} (I),
where

R₁, R₂ both represent a hydrogen atom,

L represents a linear, two-band C₁-C₆-alkylene group, in an exemplary embodiment a propylene group (—CH₂—CH₂—CH₂—) or an ethylene group (—CH₂—CH₂—),

R₃, R₄ independently represent a methyl group or an ethyl group,

a stands for the number 3 and
b stands for the number 0.

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II),
where

R5, R5′, R5″ independently represent a hydrogen atom or a C₁-C₆ alkyl group,

R6, R6′ and R6″ independently represent a C₁-C₆ alkyl group,

A, A′, A″, A′″ and A″″ independently represent a linear or branched C₁-C₂₀ divalent alkylene group,

R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (III)

-(A″″)-Si(R₆″)_{d″}(OR₅″)_{c}″ (III),
c, stands for an integer from 1 to 3,
d stands for the integer 3-c,
c′ stands for an integer from 1 to 3,
d′ stands for the integer 3-c′,
c″ stands for an integer from 1 to 3,
d″ stands for the integer 3-c″,
e stands for 0 or 1,
f stands for 0 or 1,
g stands for 0 or 1,
h stands for 0 or 1,
provided that at least one of e, f, g, and h is different from 0.

The substituents R₁, R₂, R₃, R₄, R₅, R₅′, R₅″, R₆, R₆′, R₆″, R₇, R₈, L, A′, A″″ and A″″ in the compounds of formula (I) and (II) are explained below as examples:

Examples of a C₁-C₆ alkyl group are the groups methyl, ethyl, propyl, isopropyl, n-butyl, s-butyl, and t-butyl, n-pentyl and n-hexyl. Propyl, ethyl, and methyl are suitable alkyl radicals. Examples of a C₂-C₆ alkenyl group are vinyl, allyl, but-2-enyl, but-3-enyl and isobutenyl, suitable C₂-C₆ alkenyl radicals are vinyl and allyl. Suitable examples of a hydroxy C₁-C₆ alkyl group are a hydroxymethyl, a 2-hydroxyethyl, a 2-hydroxypropyl, a 3-hydroxypropyl, a 4-hydroxybutyl group, a 5-hydroxypentyl and a 6-hydroxyhexyl group; a 2-hydroxyethyl group is particularly suitable. Examples of an amino C₁-C₆ alkyl group are the aminomethyl group, the 2-aminoethyl group, the 3-aminopropyl group. The 2-aminoethyl group is particularly suitable. Examples of a linear two-band C₁-C₂₀ alkylene group include the methylene group (—CH₂—), the ethylene group (—CH₂—CH₂—), the propylene group (—CH₂—CH₂—CH₂—) and the butylene group (—CH₂—CH₂—CH₂—). The propylene group (—CH₂—CH₂—CH₂—) is particularly suitable. From a chain length of 3 C atoms, divalent alkylene groups can also be branched. Examples of branched two-band C₃-C₂₀ alkylene groups are (—CH₂—CH(CH₃)—) and (—CH₂—CH(CH₃)—CH₂—).

In the organic silicon compounds of the formula (I)

R₁R₂N-L-Si(OR₃)ₐ(R₄)_{b} (I),

the radicals R₁ and R₂ independently of one another represent a hydrogen atom or a C₁-C₆ alkyl group. In particular, the radicals R₁ and R₂ both represent a hydrogen atom.

In the middle part of the organic silicon compound is the structural unit or the linker -L- which stands for a linear or branched, two-band C₁-C₂₀ alkylene group.

In an exemplary embodiment, -L- represents a linear, divalent C₁-C₂₀ alkylene group. Further in an exemplary embodiment, -L- represents a linear divalent C₁-C₆ alkylene group. Particularly suitable -L stands for a methylene group (—CH₂—), an ethylene group (—CH₂—CH₂—), propylene group (—CH₂—CH₂—CH₂—) or butylene (—CH₂—CH₂—CH₂—CH₂—). L stands for a propylene group (—CH₂—CH₂—CH₂—)

The organic silicon compounds of formula (I)

R₁R₂N-L-Si(OR₃)ₐ(R₄)_{b} (I),

one end of each carries the silicon-containing group —Si(OR₃)ₐ(R₄)_{b}

In the terminal structural unit —Si(OR₃)ₐ(R₄)_{b}, R₃ is hydrogen or C₁-C₆ alkyl group, and R₄ is C₁-C₆ alkyl group. R₃ and R₄ independently of each other represent a methyl group or an ethyl group.

Here a stands for an integer from about 1 to about 3, and b stands for the integer 3-a. If a stands for the number 3, then b is equal to 0. If a stands for the number 2, then b is equal to 1. If a stands for the number 1, then b is equal to 2.

The best protection against the negative effects of water and/or air pollution (“anti-pollution” effect) and the best care of stressed hair could be obtained if the agent for treating a keratinous material contains at least one organic silicon compound of formula (I) in which the radicals R₃, R₄ independently represent a methyl group or an ethyl group.

Particularly well-suited organic silicon compounds of formula (I) are
(3-Aminopropyl)triethoxysilan

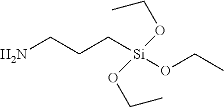

(3-Aminopropyl)trimethoxysilane

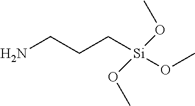

1-(3-Aminopropyl)silantriol

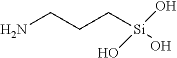

(2-Aminoethyl)triethoxysilan

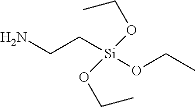

(2-Aminoethyl)trimethoxysilane

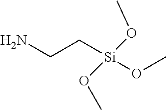

1-(2-Aminoethyl)silantriol

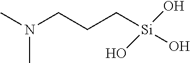

(3-Dimethylaminopropyl)triethoxysilan

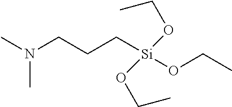 [SMILES: CCO[Si](CCCN(C)C)(OCC)OCC]

(3-Dimethylaminopropyl)trimethoxysilane

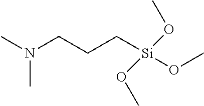

1-(3-Dimethylaminopropyl)silantriol

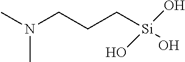

(2-Dimethylaminoethyl)triethoxysilan.

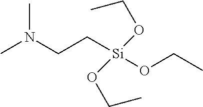

(2-Dimethylaminoethyl)trimethoxysilane and/or

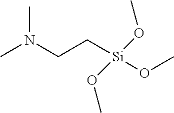

1-(2-Dimethylaminoethyl)silantriol

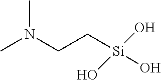

The organic silicon compound of formula (I) is commercially available. (3-aminopropyl)trimethoxysilane, for example, can be purchased from Sigma-Aldrich. (3-Aminopropyl)triethoxysilane is also commercially available from Sigma-Aldrich.

In another embodiment, the composition for treating a keratinous material includes at least one organic silicon compound of formula (II)

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II),

The organosilicon compounds of formula (II) each bear at their two ends the silicon-containing groupings (R₅O)_{c}(R₆)_{d}Si— and —Si(R₆′)_{d′}(OR₅′)_{c′}.

In the central part of the molecule of formula (II) there are the groups -(A)ₑ- and —[NR₇-(A′)]_{f}- and —[O-(A″)]_{g}- and —[NR₈-(A′″)]ₕ-. Here, each of the radicals e, f, g, and h can independently of one another stand for the number 0 or 1, with the proviso that at least one of the radicals e, f, g, and h is different from 0. In other words, an organic silicon compound of formula (II) contains at least one grouping selected from the group including -(A)- and —[NR₇-(A′)]- and —[O-(A″)]-and-[NR₈-(A′″)]-.

In the two terminal structural units (R₅O)_{c}(R₆)_{d}Sii- and —Si(R₆′)_{d′}(OR₅′)_{c}, the radicals R5, R5′, R5″ independently of one another represent a hydrogen atom or a C₁-C₆ alkyl group. The radicals R₆, R₆′ and R₆″ independently represent a C₁-C₆ alkyl group.

Here a stands for an integer from 1 to 3, and d stands for the integer 3-c. If c stands for the number 3, then d is equal to 0. If c stands for the number 2, then d is equal to 1. If c stands for the number 1, then d is equal to 2.

Analogously c′ stands for a whole number from 1 about to about 3, and d′ stands for the whole number 3-c′. If c′ stands for the number 3, then d′ is 0. If c′ stands for the number 2, then d′ is 1. If c′ stands for the number 1, then d′ is 2.

An extremely high anti-pollution effect of the agent for the treatment of a keratinous material could be obtained when the residues c and c′ both stand for the number 3. In this case d and d′ both stand for the number 0.

In another suitable one, the agent for treating a keratinous material includes at least one organic silicon compound of formula (II)

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II),

where

R₅ and R₅′ independently represent a methyl group or an ethyl group,

c and c′ both stand for the number 3 and

d and d′ both stand for the number 0.

When c and c′ both represent the number 3 and d and d′ both represent the number 0, the organic silicon compounds correspond to formula (IIa)

(R₅O)₃Si-(A)ₑ-[NR₇-(A′)]_{f}-[O-(A″)]_{g}-[NR₈-(A′″)]ₕ-Si(OR₅′)₃ (IIa).

The radicals e, f, g, and h can independently stand for the number 0 or 1, whereby at least one radical from e, f, g, and h is different from zero. The abbreviations e, f, g, and h thus define which of the groupings -(A)ₑ- and -[NR7-(A′)]_{f}- and —[O-(A″)]_{g}- and -[NR8-(A′″)]ₕ- are in the middle part of the organic silicon compound of formula (II).

In this context, the presence of certain groupings has proven to be particularly beneficial in terms of increasing the “anti-pollution” effect. Particularly good results were obtained when at least two of the residues e, f, g, and h stand for the number 1. Especially suitable e and f both stand for the number 1. Furthermore, g and h both stand for the number 0.

When e and f are both 1 and g and h are both 0, the organic silicon compounds are represented by the formula (IIb)

(R₅O)_{c}(R₆)_{d}Si-(A)-[NR₇-(A′)]-Si(R₆′)_{d′}(OR₅′V (IIb).

The radicals A, A′, A″, A′″ and A″″ independently represent a linear or branched two band C₁-C₂₀ alkylene group. In an exemplary embodiment the radicals A, A′, A″, A′″ and A″″ independently of one another represent a linear, two band C₁-C₂₀ alkylene group. Further in an exemplary embodiment the radicals A, A′, A″, A′″ and A″″ independently represent a linear two band C₁-C₆ alkylene group. In particular, the radicals A, A′, A″, A′″ and A″″ independently of one another represent a methylene group (—CH₂—), an ethylene group (—CH₂—CH₂—), a propylene group (—CH₂—CH₂—CH₂—) or a butylene group (—CH₂—CH₂—CH₂—CH₂—). In particular, the residues A, A′, A″, A′″ and A″″ stand for a propylene group (—CH₂—CH₂—CH₂—).

When the radical f represents the number 1, the organic silicon compound of formula (II) contains a structural grouping —[NR₇-(A′)]-.

When the radical h represents the number 1, the organic silicon compound of formula (II) contains a structural grouping —[NR₈-(A′″)]-.

Wherein R₇ and R₇ independently represent a hydrogen atom, a C₁-C₆ alkyl group, a hydroxy-C₁-C₆ alkyl group, a C₂-C₆ alkenyl group, an amino-C₁-C₆ alkyl group or a group of the formula (III)

(A″″)-Si(R₆″)_{d}″(OR₅″)_{c}″ (III).

For example, R₇ and R₈ independently represent a hydrogen atom, a methyl group, a 2-hydroxyethyl group, a 2-alkenyl group, a 2-aminoethyl group or a group of formula (III).

When the radical f represents the number 1 and the radical h represents the number 0, the organic silicon compound contains the grouping [NR₇-(A′)] but not the grouping —[NR₈-(A″″)]. If the radical R7 now stands for a grouping of the formula (III), the agent for treating a keratinous material contains an organic silicon compound with 3 reactive silane groups.

In another suitable one, the agent for treating a keratinous material includes at least one organic silicon compound of formula (II)

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II),

where

e and f both stand for the number 1,

g and h both stand for the number 0,

A and A′ independently represent a linear, two-band C₁-C₆ alkylene group and

R₇ represents a hydrogen atom, a methyl group, a 2-hydroxyethyl group, a 2-alkenyl group, a 2-aminoethyl group or a group of formula (III).

In another suitable embodiment, the composition for treating a keratinous material includes at least one organic silicon compound of the formula (II), wherein

e and f both stand for the number 1,

g and h both stand for the number 0,

A and A′ independently of one another represent a methylene group (—CH₂—), an ethylene group (—CH₂—CH₂—) or a propylene group (—CH₂—CH₂—CH₂), and

R₇ represents a hydrogen atom, a methyl group, a 2-hydroxyethyl group, a 2-alkenyl group, a 2-aminoethyl group or a group of formula (III).

Organic silicon compounds of formula (II) that are well suited for solving the problem are
3-(trimethoxysilyl)-N-[3-(trimethoxysilyl)propyl]-1-propanamine

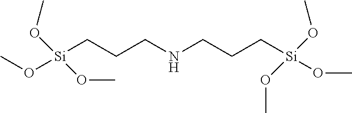

3-(Triethoxysilyl)-N-[3-(triethoxysilyl)propyl]-1-propanamine

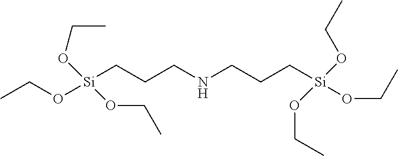 [SMILES: CCO[Si](CCCNCCC[Si](OCC)(OCC)OCC)(OCC)OCC]

N-methyl-3-(trimethoxysilyl)-N-[3-(trimethoxysilyl)propyl]-1-propanamine

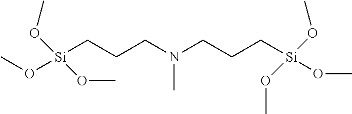

N-Methyl-3-(triethoxysilyl)-N-[3-(triethoxysilyl)propyl]-1-propanamine

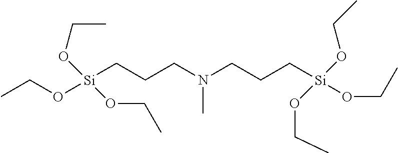 [SMILES: CCO[Si](CCCN(C)CCC[Si](OCC)(OCC)OCC)(OCC)OCC]

2-[Bis[3-(trimethoxysilyl)propyl]amino]-ethanol

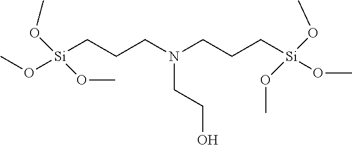

2-[bis[3-(triethoxysilyl)propyl]amino]ethanol

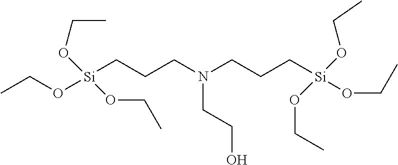 [SMILES: CCO[Si](CCCN(CCO)CCC[Si](OCC)(OCC)OCC)(OCC)OCC]

3-(Trimethoxysilyl)-N,N-bis[3-(trimethoxysilyl)propyl]-1-propanamine

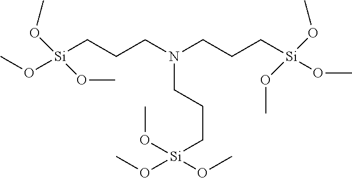

3-(Triethoxysilyl)-N,N-bis[3-(triethoxysilyl)propyl]-1-propanamine

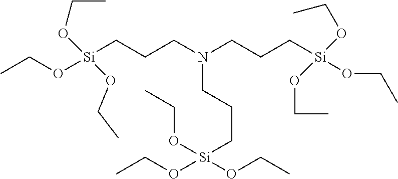 [SMILES: CCO[Si](CCCN(CCC[Si](OCC)(OCC)OCC)CCC[Si](OCC)(OCC)OCC)(OCC)OCC]

N1,N1-Bis[3-(trimethoxysilyl)propyl]-1,2-ethanediamine,

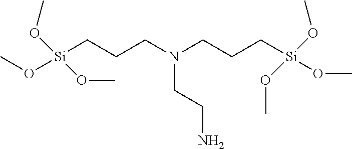

N1,N1-Bis[3-(triethoxysilyl)propyl]-1,2-ethanediamine,

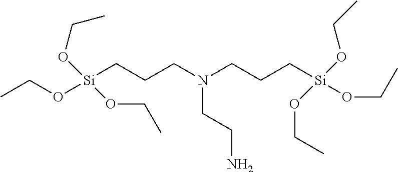 [SMILES: CCO[Si](CCCN(CCN)CCC[Si](OCC)(OCC)OCC)(OCC)OCC]

N,N-Bis[3-(trimethoxysilyl)propyl]-2-propen-1-amine

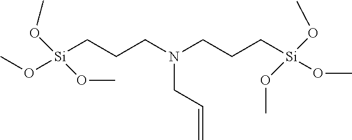

N,N-Bis[3-(triethoxysilyl)propyl]-2-propen-1-amine

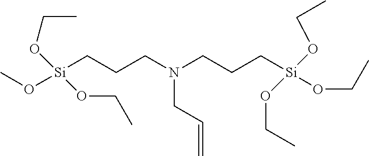 [SMILES: C=CCN(CCC[Si](OC)(OCC)OCC)CCC[Si](OCC)(OCC)OCC]

The organic silicon compounds of formula (II) are commercially available.

Bis(trimethoxysilylpropyl)amine with the CAS number 82985-35-1 can be purchased from Sigma-Aldrich.

Bis[3-(triethoxysilyl)propyl]amine, also known as 3-(triethoxysilyl)-N-[3-(triethoxysilyl)propyl]-1-propanamine, with CAS number 13497-18-2 kcan be purchased, for example, from Sigma-Aldrich or is commercially available under the product name Dynasylan 1122 from Evonik.

N-methyl-3-(trimethoxysilyl)-N-[3-(trimethoxysilyl)propy 1]-1-propanamine is alternatively referred to as Bis(3-trimethoxysilylpropyl)-N-methylamine and can be purchased commercially from Sigma-Aldrich or Fluorochem.

3-(triethoxysilyl)-N,N-bis[3-(triethoxysilyl)propyl]-1-propanamine with the CAS number 18784-74-2 can be purchased for example from Fluorochem or Sigma-Aldrich.

It has also been found to be advantageous when the agent for treating a keratinous material applied to the hair contains at least one organic silicon compound of formula (IV)

R₉Si(OR₁₀)ₖ(R₁₁)ₘ (IV).

The compounds of formula (IV) are organic silicon compounds selected from silanes having one, two or three silicon atoms, the organic silicon compound comprising one or more hydroxyl groups and/or hydrolysable groups per molecule.

The organic silicon compound(s) of formula (IV) may also be referred to as silanes of the alkylalkoxysilane or alkylhydroxysilane type,

R₉Si(OR₁₀)ₖ(R₁₁)ₘ (IV),

where

R₉ represents a C₁-C₁₂ alkyl group,

R₁₀ represents a hydrogen atom or a C₁-C₆ alkyl group,

R₁₁ represents a C₁-C₆ alkyl group

k is an integer from 1 to 3, and

m stands for the integer 3-k.

In a further suitable embodiment, the composition for treating a keratinous material contains, in addition to the organic silicon compound or compounds of formula (I), at least one further organic silicon compound of formula (IV)

R₉Si(OR₁₀)ₖ(R₁₁)ₘ (IV).

where

R₉ represents a C₁-C₁₂ alkyl group,

R₁₀ represents a hydrogen atom or a C₁-C₆ alkyl group,

R₁₁ represents a C₁-C₆ alkyl group

k is an integer from 1 to 3, and

m stands for the integer 3-k.

In a likewise suitable embodiment, the composition for treating a keratinous material contains, in addition to the organic silicon compound or compounds of the formula (II), at least one further organic silicon compound of the formula (IV) contains

R₉Si(OR₁₀)ₖ(R₁₁)ₘ (IV),

where

R₉ represents a C₁-C₁₂ alkyl group,

R₁₀ represents a hydrogen atom or a C₁-C₆ alkyl group,

R₁₁ represents a C₁-C₆ alkyl group

k is an integer from 1 to 3, and

m stands for the integer 3-k.

In another suitable embodiment, the composition for treating a keratinous material contains, in addition to the organic silicon compounds of the formula (I) and (II), at least one further organic silicon compound of the formula (IV)

R₉Si(OR₁₀)ₖ(R₁₁)ₘ (IV),

where

R₉ represents a C₁-C₁₂ alkyl group,

R₁₀ represents a hydrogen atom or a C₁-C₆ alkyl group,

R₁₁ represents a C₁-C₆ alkyl group

k is an integer from 1 to 3, and

m stands for the integer 3-k.

In the organic silicon compounds of formula (IV), the radical R₉ represents a C₁-C₁₂ alkyl group. This C₁-C₁₂ alkyl group is saturated and can be linear or branched. In an exemplary embodiment R9 stands for a linear C₁-C₈ alkyl group. In an exemplary embodiment R₉ stands for a methyl group, an ethyl group, an n-propyl group, an n-butyl group, an n-pentyl group, an n-hexyl group, an n-octyl group or an n-dodecyl group. In an example, R₉ represents a methyl group, an ethyl group or an n-octyl group.

In the organic silicon compounds of formula (IV), the radical R₁₀ represents a hydrogen atom or a C₁-C₆ alkyl group. In an example, R₁₀ represents a methyl group or an ethyl group.

In the organic silicon compounds of formula (IV), the radical R₁₁ represents a C₁-C₆ alkyl group. In an example, R₁₁ represents a methyl group or an ethyl group.

Furthermore, k stands for a whole number from about 1 to about 3, and m stands for the whole number 3-k. If k stands for the number 3, then m is equal to 0. If k stands for the number 2, then m is equal to 1. If k stands for the number 1, then m is equal to 2.

An extremely high “anti-pollution” effect could be obtained if the agent for treating a keratinous material contains at least one organic silicon compound of formula (IV) in which the radical k represents the number 3. In this case the rest m stands for the number 0.

Organic silicon compounds of the formula (IV) that are particularly suitable for solving the problem are
Methyltrimethoxysilane

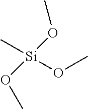

Methyltriethoxysilane

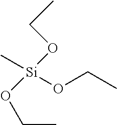

Ethyltrimethoxysilane

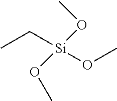

Ethyltriethoxysilane

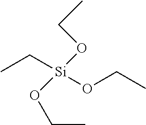

n-Hexyltrimethoxysilane

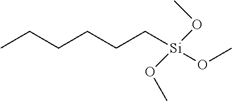

n-Hexyltriethoxysilane

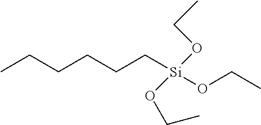 [SMILES: CCCCCC[Si](OCC)(OCC)OCC]

n-Octyltrimethoxysilane

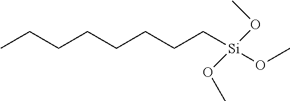

n-Octyltriethoxysilane

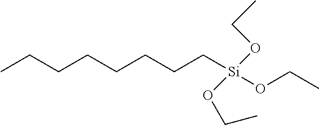 [SMILES: CCCCCCCC[Si](OCC)(OCC)OCC]

n-dodecyltrimethoxysilane and/or

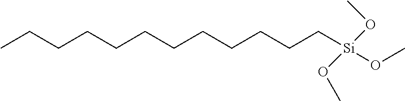 [SMILES: CCCCCCCCCCCC[Si](OC)(OC)OC]

n-dodecyltriethoxysilane.

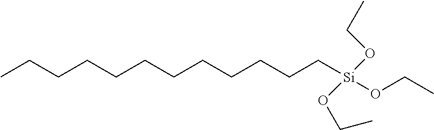 [SMILES: CCCCCCCCCCCC[Si](OCC)(OCC)OCC]

and propyltrimethoxysilane, propyltriethoxysilane, octadecyltrimethoxysilane and/or octadecyltriethoxysilane.

The organic silicon compounds described above are reactive compounds.

It was found that particularly stable and uniform films could be obtained on the keratinous material even when the agent contained two structurally different organic silicon compounds.

In a suitable embodiment, an agent exemplified includes at least one organic silicone compound of formula (I) and at least one organic silicone compound of formula (IV).

In an explicitly very particularly suitable embodiment, an agent exemplified contains at least one organic silicone compound of formula (I) selected from the group including (3-aminopropyl)triethoxysilane and (3-aminopropyl)trimethoxysilane, and additionally containing at least one organic silicone compound of formula (IV) selected from the group including methyltrimethoxysilane, methyltriethoxysilane, ethyltrimethoxysilane, ethyltriethoxysilane, propyltrimethoxysilane, propyltriethoxysilane, hexyltrimethoxysilane and hexyltriethoxysilane.

In another suitable embodiment, an agent is exemplified in that the agent—contains, based on the total weight of the agent-:

0.5 to 5 weight % of at least one first organic silicon compound selected from the group of (3-aminopropyl)trimethoxysilane, (3-aminopropyl)triethoxysilane, (2-aminoethyl)trimethoxysilane, (2-aminoethyl)triethoxysilane, (3-dimethylaminopropyl)trimethoxysilane, (3-dimethylaminopropyl)triethoxysilane (2-dimethylaminoethyl)trimethoxysilane and (2-dimethylaminoethyl)triethoxysilane, and

3.2 to 10 wt. % of at least one second organic silicon compound selected from the group including methyltrimethoxysilane, methyltriethoxysilane, ethyltrimethoxysilane, ethyltriethoxysilane, propyltrimethoxysilane, propyltriethoxysilane, hexyltrimethoxysilane, hexyltriethoxysilane, octyltrimethoxysilane, octyltriethoxysilane, dodecyltrimethoxysilane, dodecyltriethoxysilane, octyldecyltrimethoxysilane and octyldecyltriethoxysilane.

Even the addition of small amounts of water leads to hydrolysis in organic silicon compounds with at least one hydrolyzable group. The hydrolysis products and/or organic silicon compounds having at least one hydroxy group may react with each other in a condensation reaction. For this reason, both the organosilicon compounds having at least one hydrolyzable group and their hydrolysis and/or condensation products may be present in the composition. When organosilicon compounds having at least one hydroxyl group are used, both the organic silicon compounds having at least one hydroxyl group and their condensation products may be present in the composition.

A condensation product is understood to be a product formed by the reaction of at least two organic silicon compounds each having at least one hydroxyl group or hydrolyzable group per molecule with elimination of water and/or with elimination of an alkanol. The condensation products can be, for example, dimers, but also trimers or oligomers, with the condensation products being in equilibrium with the monomers. Depending on the amount of water used or consumed in the hydrolysis, the equilibrium shifts from monomeric organic silicon compounds to condensation product.

In the context of the present invention, figures in wt. % are—unless otherwise stated—always based on the total weight of the cosmetic product.

As a second ingredient essential to the invention, the cosmetic composition for treating a keratinous material contains a cationic surfactant. In the course of the work leading to this invention, it has been found that to achieve a particularly good care effect, it is particularly advantageous if the organic silicon compounds, for example (3-aminopropyl)trimethoxysilane or (3-aminopropyl)triethoxysilane, are combined with a cationic surfactant.

The combination of the at least one organic silicon compound and a cationic surfactant forms a layer on the hair. This ensures that oxidative hair colors are significantly protected from washing out. Furthermore, the hair surface is re-hydrophobized in oxidatively damaged hair, which leads to the reduction of frizz. In addition, the combability of the hair is improved.

According to a suitable embodiment of the present invention, the amount of cationic surfactant is about 0.1 to about 30% by weight, in an exemplary embodiment from about 0.5 to about 20% by weight, for example from about 1 to about 10% by weight, based on the total weight of the cosmetic composition.

According to a suitable embodiment of the present invention, the cationic surfactant includes a hydrophobic head group with a cationic charge and one or two hydrophobic end portions, wherein the hydrophobic end portion(s) represent straight-chain or branched, saturated or mono- or polyunsaturated alkyl groups, in an exemplary embodiment having a chain length of C6 to C30, for example C8 to C26, in an example C10 to C22. According to another suitable embodiment, the cationic surfactant has an ester function, an ether function, a ketone function, an alcohol function, or an amide function.

According to further suitable embodiments of the present invention, the cosmetic composition includes at least one cationic surfactant of formula (V),

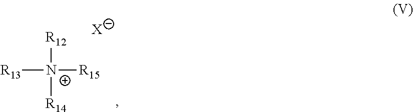

wherein
R₁₂, R₁₃, R₁₄ independently represent a C1-C6 alkyl group, a C2-C6 alkenyl group or a C2-C6 hydroxyalkyl group,
R₁₅ is a C8-C28 alkyl group, in an exemplary embodiment a C10-C22 alkyl group, and
X— represents a physiologically compatible anion,
and/or the cosmetic composition includes at least one cationic surfactant of the formula (VI),

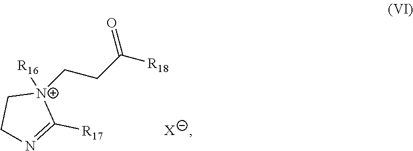

wherein
R₁₆ represents a C1-C6 alkyl group
R₁₇, R₁₈ independently represent a C7-C27 alkyl group, in an exemplary embodiment a C10-C22 alkyl group, and
X— represents a physiologically compatible anion,
and/or the cosmetic composition includes at least one cationic surfactant of the formula (VII),

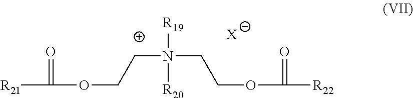

wherein
R₁₉, R₂₀ independently represent a C1-C6 alkyl group or a C2-C6 hydroxyalkyl group,
R₂₁, R₂₂ independently represent a C7-C27 alkyl group, in an exemplary embodiment a C10-C22 alkyl group, and
X— represents a physiologically compatible anion,
and/or the cosmetic composition includes at least one cationic surfactant of the formula (VIII),

NR₂₃R₂₄R₂₅ (VIII)

wherein
R₂₃, R₂₄ independently represent a C1-C6 alkyl group, a C2-C6 alkenyl group or a C2-C6 hydroxyalkyl group, and
R₂₅ represents a C8-C28 alkyl group, in an exemplary embodiment a C10-C22 alkyl group,

The cationic surfactants of formula (VIII) are amine derivatives, so-called pseudoquats. The organic radicals R₂₃, R₂₄ and R₂₅ are directly bonded to the nitrogen atom. In the acidic pH range, these are cationized, i.e., the nitrogen atom is then protonated. The physiologically compatible counterions are suitable as counterions. Steamidopropyl dimethylamine is particularly suitable among the cationic surfactants of formula (VIII).

According to a suitable embodiment of the present invention, the cosmetic composition further includes c) a nonionic surfactant. This in an exemplary embodiment includes a nonionic surfactant selected from the group including.

Alkylglucamide comprising a saturated or unsaturated, branched, or unbranched C₆ to C₂₂, in an exemplary embodiment C₁₀ to C₁₈, for example C₁₂ to C₁₆ alkyl group,

Alkyl fructoside comprising a saturated or unsaturated, branched, or unbranched C₆ to C₂₂, in an exemplary embodiment C₁₀ to C₁₈, for example C₁₂ to C₁₆ alkyl group,

An alkyl glucoside comprising a saturated or unsaturated, branched, or unbranched C₆ to C₂₂, in an exemplary embodiment C₁₀ to C₁₈, for example C₁₂ to C₁₆ alkyl group, and

Alkyl alcohol alkoxylate of the formula R₁₀(OR₁₁)ₘOH, in which R₁₀ represents a linear or branched C₆-C₂₂, in an exemplary embodiment C₁₀-C₁₈, for example C₁₂-C₁₆ alkyl group, R₁₁ represents a C₂-C₄, in an exemplary embodiment a C₂ alkyl group, and m represents 1 to 10, in an exemplary embodiment 2 to 6, for example 2 to 6,

According to a suitable embodiment of the present invention, the cosmetic composition contains two structurally different surfactants. It is particularly suitable that the cosmetic agent contains two structurally different surfactants from one another, in an exemplary embodiment the cosmetic agent contains two structurally different cationic surfactants from one another, or the cosmetic agent contains a cationic surfactant and a nonionic surfactant.

In particular, the features for treating a keratinous material may include a features for cleaning a keratinous material, a features for maintaining a keratinous material, a features for maintaining and cleaning a keratinous material, and/or a features for temporarily reshaping a keratinous material.

In the following, further ingredients of the hair treatment products are described, which may be contained in the products in addition to the previously described mandatory ingredients.

It may be suitable that the agent for treating a keratinous material further includes from 0.001 to 20% by weight of at least one quaternary compound. This applies to agents for the care of a keratinous material and agents for the care and cleaning of a keratinous material.

It is suitable that the at least one quaternary compound is selected from at least one of the groups including:

Amidoamines and/or cationized amidoamines and/or

Poly(methacryloyloxyethyltrimethylammonium compounds) and/or;

quaternized cellulose derivatives, polyquaternium 10, polyquaternium-24, polyquaternium-27, polyquaternium-67, polyquaternium-72, and/or

cationic alkyl polyglycosides and/or

cationized honey and/or

cationic guar derivatives and/or

Chitosan and/or

polymeric dimethyldiallylammonium salts and copolymers thereof with esters and amides of acrylic acid and methacrylic acid, in particular polyquaternium-7 and/or

Copolymers of vinylpyrrolidone with quaternized derivatives of dialkylaminoalkyl acrylate and methacrylate, especially polyquaternium-11 and/or

vinylpyrrolidone-vinylimidazolium methochloride copolymers, in particular polyquaternium-16 and/or

quaternized polyvinyl alcohol and/or

Polyquaternium-74,

and mixtures thereof.

It is particularly suitable that the hair treatment composition contains a cationic homopolymer falling under the INCI designation polyquaternium-37 as quaternary compounds.

It may be suitable that the agent for treating a keratinous material further includes a firming compound, in an exemplary embodiment selected from the group including waxes, synthetic polymers, and mixtures thereof.

To meet the different requirements for agents for the treatment of a keratinous material in the form of an agent for the temporary reshaping of a keratinous material (=styling agent), many synthetic polymers have already been developed as strengthening compounds, which can be used in the agent for the treatment of a keratinous material. Alternatively, or complementarity, waxes are used as strengthening compounds. Ideally, the polymers and/or waxes, when applied to the keratinous material, result in a polymer film or sheet that, on the one hand, gives the hairstyle a strong hold, but, on the other hand, is sufficiently flexible not to break when stressed.

The synthetic polymers can be divided into cationic, anionic, nonionic, and amphoteric strengthening polymers.

Suitable synthetic polymers include, for example, polymers with the following INCI designations: Acrylamides/Ammonium Acrylate Copolymer, Acrylamides/DMAPA Acrylates/Methoxy PEG Methacrylate Copolymer, Acrylamidopropyltrimonium Chloride/Acrylamide Copolymer, Acrylamidopropyltrimonium Chloride/Acrylates Copolymer, Acrylates/Acetoacetoxyethyl Methacrylate Copolymer, Acrylates/Acrylamide Copolymer, Acrylates/Ammonium Methacrylate Copolymer, Acrylates/Ammonium Methacrylate Copolymer, Acrylates/t-Butyl Acrylamide Copolymer, Acrylates Copolymer, Acrylates/C1-2 Succinates/Hydroxyacrylates Copolymer, Acrylates/Lauryl Acrylate/Stearyl Acrylate/Ethylamine Oxide Methacrylate Copolymer, Acrylates/Octylacrylamide Copolymer, Acrylates/Octylacrylamide/Diphenyl Amodimethicone Copolymer, Acrylates/Stearyl Acrylate/Ethylamine Oxide Methacrylate Copolymer, Acrylates/VA Copolymer, Acrylates/Hydroxyesters Acrylates Copolymer, Acrylates/VP Copolymer, Adipic Acid/Diethylenetriamine Copolymer, Adipic Acid/Dimethylaminohydroxypropyl Diethylenetriamine Copolymer, Adipic Acid/Epoxypropyl Diethylenetriamine Copolymer, Adipic Acid/Isophthalic Acid/Neopic Acid ntyl Glycol/Trimethylolpropane Copolymer, Allyl Stearate/VA Copolymer, Aminoethylacrylate Phosphate/Acrylates Copolymer, Aminoethylpropanediol-Acrylates/Acrylamide Copolymer, Aminoethylpropanediol-AMPD-Acrylates/Diacetone Acrylamide Copolymer, Ammonium-Acrylates/VA/Acrylates Copolymer, AMPD-Acrylates/Acrylamides Copolymer, AMPD-Acrylates/Acrylamides/Allyl Methacrylate Copolymer, AMP-Acrylates/C1-18 Alkyl Acrylates/C1-8 Alkyl Acrylamide Copolymer, AMP-Acrylates/Diacetoneacrylamide Copolymer, AMP-Acrylates/Dimethylaminoethylmethacrylate Copolymer, Bacillus/Rice Bran Extract/Soybean Extract Ferment Filtrate, Bis-Butyloxyamodimethicone/PEG-60 Copolymer, Butyl Acrylate/Ethylhexyl Methacrylate Copolymer, Butyl Acrylate/Hydroxypropyl Dimethicone Acrylate Copolymer, Butylated PVP, Butyl Ester of Ethylene/MA Copolymer, Butyl Ester of PVM/MA Copolymer, Calcium/Sodium PVM/MA Copolymer, Corn Starch/Acrylamide/Sodium Acrylate Copolymer, Diethylene Glycolamine/Epichlorohydrin/Piperazine Copolymer, Dimethicone Crosspolymer, Diphenyl Amodimethicone, Ethyl Ester of PVM/MA Copolymer, Hydrolyzed Wheat Protein/PVP Crosspolymer, Isobutylene/Ethylmaleimide/Hydroxyethylmaleimide Copolymer, Isobutylene/MA Copolymer, Isobutylmethacrylate/Bis-hydroxypropyl Dimethicone Acrylate Copolymer, Isopropyl Ester of PVM/MA Acrylate Crosspolymer, Lauryl Methacrylate/Glycol Dimethacrylate Crosspolymer, MEA-Sulphite, Methacrylic Acid/Sodium Acrylamidomethyl Propane Sulphonate Copolymer, Methacryloyl Ethyl Betaine/Acrylates Copolymer, Octylacrylamide/Acrylates/Butylaminoethyl Methacrylate Copolymer, PEG/PPG-25/25 Dimethicone, PEG-8/SMDI Copolymer, Polyacrylamide, Polyacrylate-6, Polybeta-Alanine/Glutaric Acid Crosspolymer, Polybutylene Terephthalate, Polyester-1, Polyethylacrylate, Polyethylene Terephthalate, Polymethacryloyl Ethyl Betaine, Polypentaerythrityl Terephthalate, Polyperfluoroperhydrophen-2, Polyquaternium-2 Polyquaternium-4, Polyquaternium-5, Polyquaternium-6, Polyquaternium-7, Polyquaternium-8, Polyquaternium-9, Polyquaternium-10, Polyquaternium-11, Polyquaternium-12, Polyquaternium-13, Polyquaternium-14, Polyquaternium-15, Polyquaternium-16, Polyquaternium-17, 18, Polyquaternium-19, Polyquaternium-20, Polyquaternium-22, Polyquaternium-24, Polyquaternium-27, Polyquaternium-28, Polyquaternium-29, Polyquaternium-30, Polyquaternium-31, Polyquaternium-32, Polyquaternium-33, Polyquaternium-34, Polyquaternium-35, Polyquaternium-36, Polyquaternium-37, Polyquaternium-39, Polyquaternium-45, Polyquaternium-46, Polyquaternium-47, Polyquaternium-48, Polyquaternium-49, Polyquaternium-50, Polyquaternium-55, Polyquaternium-56, Polyquaternium-569, Polyurethane-1, Polyurethane-6, Polyurethane-10, Polyvinyl Acetate, Polyvinyl Butyral, Polyvinylcaprolactam, Polyvinylformamide, Polyvinyl Imidazolinium Acetate, Polyvinyl Methyl Ether, Potassium Butyl Ester of PVM/MA Copolymer, Potassium Ethyl Ester of PVM/MA Copolymer, PPG-70 polyglyceryl-10 Ether, PPG-12/SMDI Copolymer, PPG-51/SMDI Copolymer, PPG-10 Sorbitol, PVM/MA Copolymer, PVP, PVP/VA/Itaconic Acid Copolymer, PVP/VA/Vinyl Propionate Copolymer, Rhizobian Gum, Rosin Acrylate, Shellac, Sodium Butyl Ester of PVM/MA Copolymer, Sodium Ethyl Ester of PVM/MA Copolymer, Sodium Polyacrylate, Sterculia Urens Gum, Terephthalic Acid/Isophthalic Acid/Sodium Isophthalic Acid Sulfonate/Glycol Copolymer, Trimethylolpropane Triacrylate, Trimethylbiloxylsilicon VA/Crotonates Copolymer, VA/Crotonates/Methacryloxybenzophenone-1 Copolymer, VA/Crotonates/Vinyl Neodecanoate Copolymer, VA/Crotonates/Vinyl Propionate Copolymer, VA/DBM Copolymer, VA/Vinyl Butyl Benzoate/Crotonates Copolymer, Vinylamine/Vinyl Alcohol Copolymer, Vinyl Caprolactam/VP/Dimethylaminoethyl Methacrylate Copolymer, VP/Acrylates/Lauryl Methacrylate Copolymer, VP/Dimethylaminoethylmethacrylate Copolymer, VP/DMAPA Acrylates Copolymer, VP/Hexadecene Copolymer, VP/VA Copolymer, VP/Vinyl Caprolactam/DMAPA Acrylates Copolymer, Yeast Palmitate and Styrene/VP Copolymer. Cellulose ethers such as hydroxypropyl cellulose, hydroxyethyl cellulose and methyl hydroxypropyl cellulose are also suitable.

Also, homopolyacrylic acid (INCI: Carbomer), which is commercially available under the name Carbopol® in various forms, is suitable as a firming compound.

In an exemplary embodiment, the firming compound includes a vinylpyrrolidone-containing polymer. In an example, the firming compound includes a polymer selected from the group including polyvinylpyrrolidone (PVP), vinylpyrrolidone-vinyl acetate copolymer (VP/VA copolymer), vinyl caprolactam/VP/dimethylaminoethyl methacrylate copolymer (INCI), VP/DMAPA acrylates copolymer (INCI), and mixtures thereof.

Another suitable solidifying compound is octylacrylamide/acrylates/butylaminoethyl methacrylate copolymer (INCI), which is marketed under the name “Amphomer®” by Akzo Nobel.

Accordingly, it is particularly suitable that the firming compound includes a synthetic polymer selected from the group including polyvinylpyrrolidone (PVP), vinylpyrrolidone-vinyl acetate copolymer (VP/VA copolymer), vinyl caprolactam/VP/dimethylaminoethyl methacrylate copolymer (INCI), VP/DMAPA acrylates copolymer (INCI), octylacrylamide/acrylates/butylaminoethyl methacrylate copolymer (INCI), and mixtures thereof.

The cosmetic composition may contain, in addition or as an alternative to a synthetic polymer, at least one natural or synthetic wax having a melting point above 37° C. as a firming compound.

Natural or synthetic waxes can be solid kerosenes or isoparaffins, vegetable waxes such as candelilla wax, caranuba wax, esparto grass wax, Japan wax, cork wax, sugar cane wax, ouricury wax, montan wax, sunflower wax, fruit waxes and animal waxes such as beeswaxes and other insect waxes, Whale wax, shellac wax, wool wax and brushing grease, furthermore mineral waxes, such as ceresin and ozokerite or petrochemical waxes, such as petrolatum, kerosene waxes, microwaxes of polyethylene or polypropylene and polyethylene glycol waxes can be used. It may be advantageous to use hydrogenated or cured waxes. Chemically modified waxes, in particular hard waxes such as montan ester waxes, sasol waxes and hydrogenated jojoba waxes, can also be used.

Also suitable are the triglycerides of saturated and optionally hydroxylated C₁₆₋₃₀ fatty acids, such as hydrogenated triglyceride fats (hydrogenated palm oil, hydrogenated coconut oil, hydrogenated castor oil), glyceryl tribehenate or glyceryl tri-12-hydroxystearate.

The wax components can also be selected from the group of esters of saturated, unbranched alkanecarboxylic acids having a chain length of 22 to 44 carbon atoms and saturated, unbranched alcohols having a chain length of 22 to 44 carbon atoms, provided that the wax component or the totality of wax components are solid at room temperature. Silicone waxes, for example stearyltrimethylsilane/stearyl alcohol, may also be beneficial.

Natural, chemically modified, and synthetic waxes can be used alone or in combination. Thus, several waxes can also be used. Furthermore, several wax mixtures, possibly mixed with other additives, are also commercially available. The products sold under the designations “Special Wax 7686 OE” (a mixture of cetyl palmitate, beeswax, microcrystalline wax and polyethylene with a melting range of about 73-75° C.; manufacturer: Kahl & Co), Polywax® GP 200 (a mixture of stearyl alcohol and polyethylene glycol stearate with a melting point of about 47-51° C.; manufacturer: Croda) and “Softceresin® FL 400” (a vaseline/vaseline oil/wax mixture with a melting point of about 50-54° C.; manufacturer: Parafluid Mineral Oil Company) are examples of mixtures that can be used.

In an exemplary embodiment, the wax is selected from caranuba wax (INCI: Copemicia Cerifera Cera) Bienenwachs (INCI: Beeswax), petrolatum (INCI), microcrystalline wax and especially mixtures thereof.

Suitable blends include the combination of carnauba wax (INCI: Copemicia Cerifera Cera), petrolatum and microcrystalline wax or the combination of beeswax (INCI: Beeswax) and petrolatum.

The wax or wax components should be solid at 25° C. and should melt in the range>37° C.

The composition for treating a keratinous material in an exemplary embodiment contains the firming compound in a total amount of about 0.5 to about 50% by weight, in an exemplary embodiment from about 1 to about 40% by weight, for example from about 1.5 to about 30% by weight, even for example from about 2 to about 25% by weight, based on the total weight of the cosmetic composition.

Other suitable ingredients include nonionic polymers, anionic polymers, (further) cationic polymers, waxes, protein hydrolysates, amino acids, oligopetides, vitamins, provitamins, vitamin precursors, betaines, bioquinones, purine (derivatives), care substances, plant extracts, silicones, ester oils, UV light filters, structuring agents, thickening agents, electrolytes, pH-adjusting agents, swelling agents, colorants, anti-dandruff agents, complexing agents, opacifiers, pearlescent agents, pigments, stabilizing agents, propellants, antioxidants, perfume oils and/or preservatives.

In suitable embodiments 1 to 48, the in an exemplary embodiment used organic silicon compounds are combined with the in an exemplary embodiment used cationic surfactant in a cosmetic composition as contemplated herein. The * means that the steamidopropyl dimethylamine (INCI) used is the cationized form, i.e., it is a quaternized (and thus cationic) compound.

| | Silane compound | other ingredient |
|---|---|---|
| 1 | - (3-Aminopropyl)trimethoxysilane | Steamidopropyl Dimethylamine |
| | | (INCI)* |
| 2 | (3-Aminopropyl)triethoxysilan | Steamidopropyl Dimethylamine |
| | | (INCI)* |
| 3 | - (2-Aminoethyl)trimethoxysilane | Steamidopropyl Dimethylamine |
| | | (INCI)* |
| 4 | - (2-Aminoethyl)triethoxysilane | Steamidopropyl Dimethylamine |
| | | (INCI)* |
| 5 | - (3-Dimethylaminopropyl)trimethoxysilane | Steamidopropyl Dimethylamine |
| | | (INCI)* |
| 6 | (3-Dimethylaminopropyl)triethoxysilane | Steamidopropyl Dimethylamine |
| | | (INCI)* |
| 7 | (2-Dimethylaminoethyl)trimethoxysilane | Steamidopropyl Dimethylamine |
| | | (INCI)* |
| 8 | (2-Dimethylaminoethyl)triethoxysilane | Steamidopropyl Dimethylamine |
| | | (INCI)* |
| 9 | 3-(trimethoxysilyl)-N-[3- | Steamidopropyl Dimethylamine |
| | (trimethoxysilyl)propyl]-1-propanamine | (INCI)* |
| 10 | 3-(Triethoxysilyl)-N-[3- | Steamidopropyl Dimethylamine |
| | (triethoxysilyl)propyl]-1-propanamine | (INCI)* |
| 11 | N-methyl-3-(trimethoxysilyl)-N-[3- | Steamidopropyl Dimethylamine |
| | (trimethoxysilyl)propyl]-1-propanamine | (INCI)* |
| 12 | N-Methyl-3-(triethoxysilyl)-N-[3- | Steamidopropyl Dimethylamine |
| | (triethoxysilyl)propyl]-1-propanamine | (INCI)* |
| 13 | 2-[Bis[3-(trimethoxysilyl)propyl]amino]- | Steamidopropyl Dimethylamine |
| | ethanol | (INCI)* |
| 14 | 2-[bis[3-(triethoxysilyl)propyl]amino]ethanol | Steamidopropyl Dimethylamine |
| | | (INCI)* |
| 15 | 3-(Trimethoxysilyl)-N,N-bis[3- | Steamidopropyl Dimethylamine |
| | (trimethoxysilyl)propyl]-1-propanamine | (INCI)* |
| 16 | 3-(Triethoxysilyl)-N,N-bis[3- | Steamidopropyl Dimethylamine |
| | (triethoxysilyl)propyl]-1-propanamine | (INCI)* |
| 17 | N1,N1-Bis[3-(trimethoxysilyl)propyl]-1,2- | Steamidopropyl Dimethylamine |
| | ethanediamine | (INCI)* |
| 18 | N1,N1-Bis[3-(triethoxysilyl)propyl]-1,2- | Steamidopropyl Dimethylamine |
| | ethanediamine | (INCI)* |
| 19 | N,N-Bis[3-(trimethoxysilyl)propyl]-2-propen- | Steamidopropyl Dimethylamine |
| | 1-amine | (INCI)* |
| 20 | N,N-Bis[3-(triethoxysilyl)propyl]-2-propen-1- | Steamidopropyl Dimethylamine |
| | amine | (INCI)* |
| 21 | Methyltrimethoxysilane | Steamidopropyl Dimethylamine |
| | | (INCI)* |
| 22 | Methyltriethoxysilane | Steamidopropyl Dimethylamine |
| | | (INCI)* |
| 23 | Ethyltrimethoxysilane | Steamidopropyl Dimethylamine |
| | | (INCI)* |
| 24 | Ethyltriethoxysilane | Steamidopropyl Dimethylamine |
| | | (INCI)* |
| 25 | Propyltrimethoxysilane | Steamidopropyl Dimethylamine |
| | | (INCI)* |
| 26 | Propyltriethoxysilane | Steamidopropyl Dimethylamine |
| | | (INCI)* |
| 27 | Hexyltrimethoxysilane | Steamidopropyl Dimethylamine |
| | | (INCI)* |
| 28 | Hexyltriethoxysilane | Steamidopropyl Dimethylamine |
| | | (INCI)* |
| 29 | Octyltrimethoxysilane | Steamidopropyl Dimethylamine |
| | | (INCI)* |
| 30 | Octyltriethoxysilane | Steamidopropyl Dimethylamine |
| | | (INCI)* |
| 31 | Dodecyltrimethoxysilane | Steamidopropyl Dimethylamine |
| | | (INCI)* |
| 32 | Dodecyltriethoxysilane | Steamidopropyl Dimethylamine |
| | | (INCI)* |
| 33 | Octadecyltriethoxysilane | Steamidopropyl Dimethylamine |
| | | (INCI)* |
| 34 | Octadecyltriethoxysilane | Steamidopropyl Dimethylamine |
| | | (INCI)* |
| 35 | (3-Aminopropyl)triethoxysilane + | Steamidopropyl Dimethylamine |
| | methyltrimethoxysilane | (INCI)* |
| 36 | (3-Aminopropyl)triethoxysilane + | Steamidopropyl Dimethylamine |
| | methyltriethoxysilane | (INCI)* |
| 37 | (3-Aminopropyl)triethoxysilane + | Steamidopropyl Dimethylamine |
| | ethyltrimethoxysilane | (INCI)* |
| 38 | (3-Aminopropyl)triethoxysilane + | Steamidopropyl Dimethylamine |
| | ethyltriethoxysilane | (INCI)* |
| 39 | (3-Aminopropyl)triethoxysilane + | Steamidopropyl Dimethylamine |
| | propyltrimethoxysilane | (INCI)* |
| 40 | (3-Aminopropyl)triethoxysilane + | Steamidopropyl Dimethylamine |
| | propyltriethoxysilane | (INCI)* |
| 41 | (3-Aminopropyl)triethoxysilane + | Steamidopropyl Dimethylamine |
| | hexyltrimethoxysilane | (INCI)* |
| 42 | (3-aminopropyl)triethoxysilane + | Steamidopropyl Dimethylamine |
| | hexyltriethoxysilane | (INCI)* |
| 43 | (3-Aminopropyl)triethoxysilane + | Steamidopropyl Dimethylamine |
| | octyltrimethoxysilane | (INCI)* |
| 44 | (3-Aminopropyl)triethoxysilane + | Steamidopropyl Dimethylamine |
| | Octyltriethoxysilane | (INCI)* |
| 45 | (3-Aminopropyl)triethoxysilane + | Steamidopropyl Dimethylamine |
| | dodecyltrimethoxysilane | (INCI)* |
| 46 | (3-Aminopropyl)triethoxysilane + | Steamidopropyl Dimethylamine |
| | dodecyltriethoxysilane | (INCI)* |
| 47 | (3-aminopropyl)triethoxysilane + | Steamidopropyl Dimethylamine |
| | octyldecyltrimethoxysilane | (INCI)* |
| 48 | (3-Aminopropyl)triethoxysilane + | Steamidopropyl Dimethylamine |
| | Octyldecyltriethoxysilane | (INCI)* |

The active ingredient combination of at least one organic silicon compound and a cationic surfactant may already be present in the composition for treating a keratinous material. In this embodiment, the agent for treating a keratinous material is already distributed in a form ready for use. To provide a formulation that is as stable as possible during storage, the agent itself is in an exemplary embodiment packaged with low or no water.

Alternatively, the at least one organic silicon compound is added a maximum of 12 hours, in an exemplary embodiment a maximum of 6 hours, for example a maximum of 3 hours, even for example a maximum of 1 hour prior to application of the keratinous material treatment composition to a base comprising all the ingredients of the keratinous material treatment composition except the at least one organic silicon compound.

Furthermore, alternatively, the organic silicon compound and the cationic surfactant are added to a cosmetic product only shortly before use, i.e., 1 minute to 12 hours, in an exemplary embodiment from about 2 minutes to about 6 hours, in an example from about 1 minute to about 3 hours, especially in an exemplary embodiment from about 1 minute to about 1 hour.

In another alternative, the bis(triethoxysilylpropyl)amine is added to an aqueous solution which is applied to the hair and, in the second step, an aqueous solution or a cosmetic agent containing cationic surfactants is applied to the hair.

For example, the user may first mix or shake an agent (a) comprising the organic silicon compound(s) with an agent (β) comprising the remaining ingredients of the agent for treating a keratinous material. The user can now apply this mixture of (α) and (β)—either directly after its preparation or after a short reaction time of about 1 minute to about 20 minutes—to the keratinous materials. The agent (β) may contain water, in particular water in an amount>30% by weight, based on the total weight of the agent for treating keratinous materials.

Another object of the present application is the use of a cosmetic composition as contemplated herein for treating a keratinous material

for the care of keratinous material,
to reduce and/or prevent fading of oxidatively colored keratinous material,
for hydrophobizing the surface of keratinous material, and/or
to improve the combability of keratinous material.

Regarding further suitable embodiments of use, the same applies mutatis mutandis as to the cosmetic agents.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic composition for treating a keratinous material comprising:
a) at least one organic silicon compound; and
b) a cationic surfactant.

2. The cosmetic composition for treating a keratinous material according to claim 1,
wherein
the at least one organic silicon compound comprises a compound of the formula (I) and/or (II),
wherein in the organic silicon compound of formula (I)
R₁R₂N-L-Si(OR₃)ₐ(R₄)_{b} (I),
R₁, R₂ both represent a hydrogen atom,
L represents a linear, two-band C₁-C₆-alkylene group,
R₃, R₄ independently represent a methyl group or an ethyl group,
a stands for the number 3 and
b stands for the number 0, and
wherein in the organic silicon compound of formula (II)
(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II),
R₅, R_{5′}, R_{5″}, R₆, R_{6′} and R_{6″} independently represent a C₁-C₆ alkyl group,
A, A′, A″, A′″ and A″″ independently represent a linear or branched C₁-C₂₀ divalent alkylene group,
R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (III)
(A″″)-Si(R₆″)_{d″}(OR₅″)_{c″} (III),
c stands for an integer from 1 to 3,
d stands for the integer 3-c,
c′ stands for an integer from 1 to 3,
d′ stands for the integer 3-c′,
c″ stands for an integer from 1 to 3,
d″ stands for the integer 3-c″,
e stands for 0 or 1,
f stands for 0 or 1,
g stands for 0 or 1,
h stands for 0 or 1,
with the proviso that at least one of the residues from e, f, g, and h is different from 0.

3. The cosmetic composition for treating a keratinous material according to claim 1,
wherein
the agent for treating a keratinous material contains at least one organic silicon compound of formula (I) selected from the group of
(3-Aminopropyl)trimethoxysilane
(3-Aminopropyl)triethoxysilan
(2-Aminoethyl)trimethoxysilane
(2-Aminoethyl)triethoxysilan
(3-Dimethylaminopropyl)trimethoxysilane
(3-Dimethylaminopropyl)triethoxysilan
(2-dimethylaminoethyl)trimethoxysilane and
(2-dimethylaminoethyl)triethoxysilane,
or wherein,
the agent for treating a keratinous material contains at least one organic silicon compound of formula (II) selected from the group of
3-(trimethoxysilyl)-N-[3-(trimethoxysilyl)propyl]-1-propanamine
3-(Triethoxysilyl)-N-[3-(triethoxysilyl)propyl]-1-propanamine
N-methyl-3-(trimethoxysilyl)-N-[3-(trimethoxysilyl)propyl]-1-propanamine
N-Methy 1-3-(triethoxysilyl)-N-[3-(triethoxysilyl)propyl]-1-propanamine
2-[Bis[3-(trimethoxysilyl)propyl]amino]-ethanol
2-[Bis[3-(triethoxysilyl)propyl]amino]ethanol
3-(Trimethoxysilyl)-N,N-bis[3-(trimethoxysilyl)propyl]-1-propanamine
3-(Triethoxysilyl)-N,N-bis[3-(triethoxysilyl)propyl]-1-propanamine
N1,N1-Bis[3-(trimethoxysilyl)propyl]-1,2-ethanediamine,
N1,N1-Bis[3-(triethoxysilyl)propyl]-1,2-ethanediamine,
N,N-Bis[3-(trimethoxysilyl)propyl]-2-propen-1-amine and
N,N-Bis[3-(triethoxysilyl)propyl]-2-propen-1-amine.

4. The cosmetic composition for treating a keratinous material according to claim 1, wherein the organic silicon compound is present in the cosmetic composition in an amount of from about 0.01 to about 10% by weight, based on the total weight of the cosmetic composition, the organic silicon compound being (3-aminopropyl)triethoxysilane.

5. The cosmetic composition for treating a keratinous material according to claim 1, wherein the composition for treating a keratinous material comprises at least one organic silicon compound of formula (IV),
R₉Si(OR₁₀)ₖ(R₁₁)ₘ (IV),
which is selected from the group of
Methyltrimethoxysilane
Methyltriethoxysilane
Ethyltrimethoxysilane
Ethyltriethoxysilane
Propyltrimethoxysilane
Propyltriethoxysilane
Hexyltrimethoxysilane
Hexyltriethoxysilane
Octyltrimethoxysilane
Octyltriethoxysilane
Dodecyltrimethoxysilane
Dodecyltriethoxysilane
Octadecyltrimethoxysilane and
Octadecyltriethoxysilane.

6. The cosmetic composition for treating a keratinous material according to claim 1, wherein the cosmetic composition for treating a keratinous material comprises at least two organic silicon compounds which are structurally different from one another.

7. The cosmetic composition for treating a keratinous material according to claim 1, wherein the cationic surfactant comprises a hydrophobic head group with a cationic charge and one or two hydrophobic end parts, the hydrophobic end part or parts being straight-chain or branched, saturated or mono- or polyunsaturated alkyl groups, and having a chain length of C6 to C30, and/or wherein the cationic surfactant has an ester function, an ether function, a ketone function, an alcohol function or an amide function.

8. The cosmetic composition for treating a keratinous material according to claim 1, wherein the cosmetic composition comprises at least one cationic surfactant of formula (V), 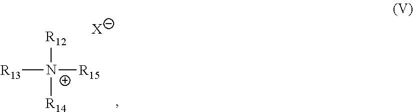
wherein
R₁₂, R₁₃, R₁₄ independently represent a C1-C6 alkyl group, a C2-C6 alkenyl group or a C2-C6 hydroxyalkyl group,
R₁₅ is a C8-C28 alkyl group, and
X— stands for a physiologically compatible anion,
and/or wherein the cosmetic composition comprises at least one cationic surfactant of the formula (VI), 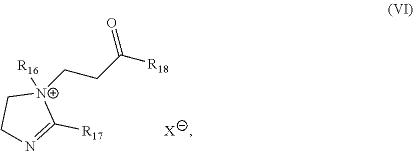
wherein
R₁₆ represents a C1-C6 alkyl group
R₁₇, R₁₈ independently represent a C7-C27 alkyl group, and
X— stands for a physiologically compatible anion,
and/or wherein the cosmetic composition comprises at least one cationic surfactant of formula (VII), 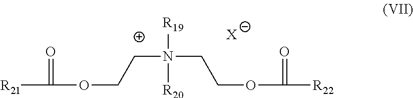
wherein
R₁₉, R₂₀ independently represent a C1-C6 alkyl group or a C2-C6 hydroxyalkyl group,
R₂₁, R₂₂ independently represent a C7-C27 alkyl group, and
X— stands for a physiologically compatible anion,
and/or wherein the cosmetic composition comprises at least one cationic surfactant of the formula (VIII)
NR₂₃R₂₄R₂₅ (VIII)
wherein
R₂₃, R₂₄ independently represent a C1-C6 alkyl group, a C2-C6 alkenyl group or a C2-C6 hydroxyalkyl group, and
R₂₅ represents a C8-C28 alkyl group,

9. The cosmetic composition for treating a keratinous material according to claim 1, wherein the cosmetic composition further comprises
(c) a nonionic surfactant,
wherein the nonionic surfactant is selected from the group of:
Alkylglucamide comprising a saturated or unsaturated, branched, or unbranched C₆ to C₂₂alkyl group,
An alkyl glucoside comprising a saturated or unsaturated, branched, or unbranched C₆ to C₂₂alkyl group,
An alkyl fructoside comprising a saturated or unsaturated, branched, or unbranched C₆ to C₂₂, and
Alkyl alcohol alkoxylate of the formula R₁₀(OR₁₁)ₘOH, in which R₁₀ represents a linear or branched C₆-C₂₂alkyl group, R₁₁ represents a C₂-C₄alkyl group, and m represents from 1 to about 10.

10. The cosmetic composition for treating a keratinous material according to claim 1, wherein the cosmetic composition comprises two structurally different surfactants.

11. The cosmetic composition for treating a keratinous material according to claim 1, wherein the cationic surfactant is present in the cosmetic composition in an amount of from about 0.1 to about 30% by weight, based on the total weight of the cosmetic composition.

12. The cosmetic composition for treating a keratinous material according to claim 1, wherein the composition for treating a keratinous material comprises based on the total weight of the composition for treating a keratinous material:
from about 0.5 to about 3 weight % of at least one first organic silicon compound selected from the group of (3-aminopropyl)trimethoxysilane, (3-aminopropyl)triethoxysilane, (2-aminoethyl)trimethoxysilane, (2-aminoethyl)triethoxysilane, (3-dimethylaminopropyl)trimethoxysilane, (3-dimethylaminopropyl)triethoxysilane (2-dimethylaminoethyl)trimethoxysilane and (2-dimethylaminoethyl)triethoxysilane, and
from about 3.2 to about 7 wt. % of at least one second organic silicon compound selected from the group of methyltrimethoxysilane, methyltriethoxysilane, ethyltrimethoxysilane, ethyltriethoxysilane, propyltrimethoxysilane, propyltriethoxysilane, hexyltrimethoxysilane, hexyltriethoxysilane, octyltrimethoxysilane, octyltriethoxysilane, dodecyltrimethoxysilane, dodecyltriethoxysilane, octadecyltrimethoxysilane and octadecyltriethoxysilane.

13. (canceled)
